(19) United States
(12) Patent Application Publication
(10) Pub. No.: US 2020/0123590 A1
(43) Pub. Date: Apr. 23, 2020

(54) SINGLE NUCLEUS AND SINGLE MOLECULE CHROMATIN INTERACTION ASSAYS

(71) Applicant: The Jackson Laboratory, Bar Harbor, ME (US)

(72) Inventors: Yijun RUAN, Farmington, CT (US); Meizhen ZHENG, Farmington, CT (US); Emaly PIECUCH, Farmington, CT (US)

(73) Assignee: The Jackson Laboratory, Bar Harbor, ME (US)

(21) Appl. No.: 16/623,076

(22) PCT Filed: Jun. 18, 2018
(86) PCT No.: PCT/US2018/038071

§ 371 Date: Dec. 16, 2019

Related U.S. Application Data
(60) Provisional application No. 62/520,665, filed on Jun. 16, 2017.

(51) Int. Cl. C12Q 1/6804

(52) U.S. Cl. CPC C12Q 1/6804; C12Q 2565/601; C12Q 2565/133; C12Q 2523/101

(57) ABSTRACT

The present invention provides a method for next generation chromatin interaction assays based on the single molecule protein-detection and DNA-sequencing platform at the single molecule and single nucleus single molecule levels. The present invention has the advantages of single molecule resolution in single nuclei and the elimination of proximity ligation and PCR amplification steps. The present invention provides revolutionary biological insights in the organization of the 3D genome and its modulation.

### CROSS REFERENCE TO RELATED APPLICATION

This application claims priority to U.S. Provisional Application No. 62/520,665, filed Jun. 16, 2017, which is hereby incorporated by reference in its entirety.

### FIELD OF THE INVENTION

The present disclosure generally relates to the field of three-dimensional (3D) genome organizational mapping and chromatin interaction. The present disclosure has the advantages of single molecule resolution and single nucleus resolution on solid phase and the elimination of proximity ligation and PCR amplification steps, avoiding technical noise. Specifically, the present disclosure provides a set of chromatin interaction assays that can detect interactions between more than two loci in a single chromatin complex and in a single nucleus. The methods are based on single molecule protein detection and commercial platform DNA sequencing. The chromatin interaction assays of this disclosure provide revolutionary biological insights in the 3D genome organization and its modulation.

### BACKGROUND

The human genome is extensively folded into 3D protein-mediated chromatin loops that provide a topological basis for genome functions, including transcriptional regulation. The current knowledge of the 3D genome organization is largely based on population level studies of chromatin molecules from millions of cells, so the current view is an average of many individual cells. Although such observations have revealed principles of 3D genome organization, they fail to provide a precise view of how the human genome is folded at the molecular level in individual chromatin complexes and individual nuclei, masking important molecular dynamics and cell-to-cell heterogeneity. For instance, previously reported chromatin interaction assay by paired-end tagged (ChIA-PET) data revealed that multiple gene promoters and enhancers could be brought together to form complex chromatin loops, suggesting a topological mechanism for gene co-regulation (Li, G., et al., Cell (2012) 148(1-2): 84-98, PMID:22265404); Tang, Z. et al., Cell (2015) 163(7): 1611-1627, PMID:26686651). However, it is not clear whether individual nuclei contain such multiplex chromatin loops or if individual loops occur differently between nuclei and collectively appear as a joint looping complex in bulk cell analysis. This is because the proximity ligation and paired-end-tag sequencing upon which ChIA-PET and the other commonly used technique for determining chromatin interactions, Hi-C, are based can only reveal paired chromatin interactions between two loci, not the contact relationships involving more than two loci.

Growing evidence for the existence of extensive genome structural stochasticity and transcriptional heterogeneity across phenotypically identical cells further confounds our interpretation of higher-order genome organization and function.

Indeed, there are active efforts in developing single cell 3D genome mapping technologies, such as single cell Hi-C(Nagano, T., et al., Nature (2013) 502(7469): 59-64, PMID:24067610). However, the current strategies based on single cell isolation or individual cell barcoding continue to rely on conventional molecular approaches for DNA manipulation, including proximity ligation, DNA amplification, and library preparation for sequencing. Such strategies are unlikely to break current technology barriers for true single molecule analysis of chromatin interactions in single cells. New technologies are urgently needed. Specifically needed are chromatin interaction assays capable of identifying interactions between more than two chromatin loci, providing data from a single chromatin complex (a single molecule), providing data single molecule data of chromatin complexes from a single nucleus, and mapping this data to provide a view of chromatin interactions across a genome.

### SUMMARY

This disclosure provides a novel technology for examining chromatin interactions in a single chromatin complex, including interactions between more than two chromatin loci and then using the data from single chromatin complexes to generate a genome-wide map of chromatin interaction. The disclosure provides an assay called single molecule Chromatin Interaction Analysis (smChIA), which may conveniently utilize a commercial platform for single molecule protein-detection and DNA sequencing. SEQ LL is an example of such a platform.

In one aspect, the present invention provides a novel sequential sequencing method for single molecule chromatin interaction analysis. Specifically, the smChIA comprises immobilizing chromatin materials on a flow cell surface for single molecule protein detection and direct sequential single molecule sequencing of DNA fragments tethered together by proteins in each chromatin complex. Notably the sequential sequencing is accomplished without chromatin proximity ligation or DNA amplification.

The present disclosure provides a method for the determination of co-localization of multiple proteins and RNA factors at each locus. Thus, smChIA has the potential to transform the field of chromatin interaction analysis and 3D genome biology.

The disclosure provides smChIA, a method of determining chromatin interactions at a single molecule level. SmChIA comprises the steps of:
a) crosslinking genomic DNA and proteins in a cell;
b) fragmenting the crosslinked genomic DNA to provide a chromatin complex, the chromatin complex containing DNA and one or more specific proteins;
c) ligating two or more different barcoded linkers to the DNA in the chromatin complex to form a barcoded chromatin complex;
d) immobilizing the barcoded chromatin complex onto a surface;
e) imaging the barcoded chromatin complex using a TIRF microscope at a single molecule level;
f) sequentially sequencing the DNA in the barcoded chromatin complex at a single molecule level to generate a plurality of sequence reads; and
g) mapping said plurality of sequence reads to a referenced genome to produce a genomic location of said sequenced reads to generate a 3D genomic connectivity map, wherein said connectivity map is indicative a physical interaction between the genomic DNA and proteins present in the chromatin complex at a single molecule level.

Prior to immobilizing the barcoded chromatin complex onto the surface, the barcoded chromatin complex can be immunoprecipitated by a first antibody capable of binding to a specific protein in the barcoded chromatin complex, so as to enrich the chromatin complexes for smChIA analysis.

The imaging step e) can include immunostaining the barcoded chromatin complex with a second antibody capable of binding to a specific protein in the barcoded chromatin complex. Following the imaging step the smChIA method can include sequentially detecting specific proteins by immunostaining with a fluorescent labeled antibody against a specific protein present in the chromatin complex. The smChIA method can also include removing the protein components from the chromatin complex, and retaining the DNA templates of the chromatin complex that are immobilized on surface.

The present disclosure also provides a method of determining a chromatin interaction in a single nucleus, said method comprising the steps of:
a) providing a single nucleus, said nucleus comprising genomic DNA and proteins;
b) crosslinking genomic DNA and proteins in the nucleus;
c) fragmenting the crosslinked genomic DNA in situ to provide a plurality of chromatin complexes, each chromatin complex containing genomic DNA and one or more specific proteins;
d) ligating two or more different barcoded linkers to the DNA in the chromatin complexes, to form barcoded chromatin complexes;
e) immobilizing said single nucleus onto a surface;
f) lysing said single nucleus such that the barcoded chromatin complexes contained in the nucleus are dispersed on the surface;
g) immunostaining the barcoded chromatin complex with an antibody capable of binding to a protein present in the barcoded chromatin complex;
h) imaging the immunostained barcoded chromatin complex using a TIRF microscope;
i) repeat g) and h) for sequential detection for two or more antibodies.
j) sequential sequencing of the barcoded chromatin complex at a single molecule level to generate a plurality of sequence reads; and
l) mapping the plurality of sequence reads to a referenced genome to produce a genomic location of the sequenced reads to generate a 3D genomic connectivity map; wherein said connectivity map is indicative a physical interaction between genomic DNA and proteins in the chromatin complex at a single molecule level. In certain embodiments steps g) and h) of this method may be repeated for sequential detection with two or more antibodies.

Various objects and advantages of this disclosure will become apparent to those skilled in the art from the following detailed description of the preferred embodiment, when read in light of the accompanying drawings and claims.

### BRIEF DESCRIPTION OF THE DRAWINGS

FIG. 1 is a schematic diagram of the smChIA method.

FIG. 1A depicts the process of chromatin complex preparation.

FIG. 1B shows chromatin complex loading onto the flow cell surface.

FIG. 1C describes the SEQ LL platform using Total Internal Reflection Fluorescence (TIRF) microscopy for single molecule protein detection and DNA sequencing.

FIG. 1D describes DNA fragments ligated with different barcoded linkers tethered in each chromatin complex are clustered in dispersed spots ˜400 nm in diameter (the optical resolution limit of TIRF) for sequential primer sequencing.

FIG. 2 illustrates the steps required to obtain chromatin complexes for the purpose of smChIA.

FIG. 3 depicts chromatin complex loading to the flow cell and the key steps required for sequential sequencing of individual chromatin complexes.

FIG. 4A depicts the read length distribution of smChIP sequencing from two test smChIP experiments by single molecule sequencing. The peak read length is at 28-29 bp for both datasets.

FIG. 4B depicts the mapping of smChIP reads to the Drosophila reference genome (dm3). An example genomic region is shown. The top two panels are the mapping alignments of smChIP reads from the two smChIP experiments, showing RNAPII binding peaks involved in chromatin interactions as identified by ChIA-PET (two lower panels).

FIG. 5A depicts a snapshot image of smChIA sequencing from the SEQ LL instrument. Each spot represents an optical point on the flow cell where fluorescent signals were captured for each nucleotide addition during the sequencing progression. Each spot may contain multiple DNA fragments to be sequentially sequenced by different primers, each primer complementary to a different barcoded linker.

FIG. 5B depicts fluorescence signal plot of a sequential sequencing runs by two primers. Specific nucleotide index of linkers are indicated.

FIG. 5C depicts examples of specific smChIA sequencing reads, their nucleotide composition, their mapping alignment in reference genome, and the corresponding ChIA-PET data.

FIG. 6 describes the barcoded biotinylated linkers and sequencing primers used in the smChIA method. The 6 bp random barcoded regions of each linker occur at the 5′ end of the biotinylated DNA oligonucleotide. The “T” nucleotide overhand that is complementary to the “A-tail” contained in the genomic DNA of the chromatin complexes, facilitating linker ligation is highlighted in bold. The respective sequencing primer for each barcoded linker is shown above the biotinylated linker. The terminal 3′ linked biotin is shown as “/3Bio” and the optional terminal linked ALEXA FLUOR 647 fluorophore is shown as “/iThioMC6D//NAlexa647-5”. Oligonucleotides are designed to contain uracil nucleotides (U), which allows for specific USER digestion. Oligonucleotides are designed such that every 5 nucleotides of the non-template strand of the oligo contain a U base. This design allows for explicit removal of part of the non-template strand, facilitating primer annealing and allowing sequential sequencing. The barcoded linkers are referred to in the claims by SEQ ID numbers. SmChIA linker 1 is SEQ ID NO: 1; smChIA linker 2 is SEQ ID NO: 2; smChIA linker 3 is SEQ ID NO: 3; smChIA linker 4 is SEQ ID NO: 4; smChIA linker 5 is SEQ ID NO: 5; smChIA linker 6 is SEQ ID NO: 6; smChIA linker 7 is SEQ ID NO: 7; and smChIA linker 8 is SEQ ID NO: 8.

FIG. 7 shows the block diagram for the smChIA method.

FIG. 8 shows the steps required to apply smChIA to single nuclei. Briefly, cells were cross-linked and their cellular membranes were removed. Nuclei remained intact, and nuclear membranes were permeabilized in order to allow restriction enzyme digestion, end repair, A-Tailing and linker ligation were done in situ. Intact nuclei were then dispersed onto a streptavidin-coated flow cell, allowed to hybridize, and their distinct 2-dimensional position on the flow cell surface were recorded. Nuclei were then lysed on the slide, allowing chromatin complexes to disperse far enough apart to be observed as discrete complexes (>400 nm) while remaining relatively close to the original position of the nucleus on the flow cell. Importantly, chromatin complexes that came from a particular nucleus remained closely grouped to each other, allowing contents from one nucleus to be distinguished from those of another nucleus.

FIG. 9 provides example images of smChIA applied to a single nucleus (single nucleus smChIA).

FIG. 9A Shows successful linker ligation in situ, performed within intact nuclei, an ALEXA 647 fluorophore labeled biotinylated linker was applied genome wide after restriction enzyme digestion, end repair and A-Tailing steps were performed.

FIG. 9B Chromatin complexes labeled with ALEXA 647 fluorophore and biotinylated linker were resolved and distinguished from one another on a streptavidin-coated flow cell.

FIG. 9C Chromatin complexes that contained the ALEXA 647 fluorophore labeled biotinylated linker and RNAPII (or any other transcription factor of interest, identified by antibody staining) were identified and distinguished from unique single nuclei by TIRF microscopy.

### DETAILED DESCRIPTION

The present disclosure can be better understood from the following description of embodiments, taken in conjunction with the accompanying drawings. It should be apparent to those skilled in the art that the described embodiments are merely illustrative and not limiting.

### Definitions

The following terms are used in the specification and claims.

The symbol “˜” is used to indicate an approximate numerical value. The level of approximation in the value will be readily apparent to one of ordinary skill of art in the relevant field.

“A-Tailing” is an enzyme-based method used to add a non-templated nucleotide to the 3′ end of a blunt, double stranded nucleic acid molecule.

A “barcoded linker” is a short (e.g. 10-50 bp) DNA sequence ligated to the free ends of chromatin DNA that is contained in a chromatin complex. The barcoded linker contains a “barcode” of 6-16 nucleotides. The barcode functions as a taxonomic tag for efficient identification of multiple genomic DNA sequences in a single chromatin complex. The remainder of the barcoded linker is linker DNA that can span the barcode and a fluorescent label or substrate ligand, such as biotin.

“ChIA-PET” is a chromatin capture technique that incorporates chromatin immunoprecipitation (ChIP)-based enrichment, proximity ligation of chromatin, PCR amplification, high-throughput sequencing, and reference genome mapping to determine long-range chromatin interactions genome-wide.

“Chromatin” is a native complex of genomic DNA and proteins found in a cell. Chromatin may also contain RNA.

“Chromatin complex” refers to a functional unit of chromatin, containing DNA, protein, and optionally RNA. Certain chromatin complexes have gene regulatory significance.

“Chromatin immunoprecipitation (ChIP) is a procedure involving crosslinking of chromatin that is used to determine whether a particular protein binds to or is localized to a specific DNA sequence in vivo.

“Crosslinking” is the chemical bonding of one polymer to another; in this case, crosslinking is used to chemically link DNA within a chromatin complex to maintain the structure of chromatin complexes during additional steps.

“Chromatin loading,” in the context of this disclosure, is the act of adding chromatin complexes to a flow-cell. The flow-cell can be coated with streptavidin in order for the hybridization of chromatin and streptavidin via the biotin molecules contained in the chromatin.

“Drosophila S2 Cell” are Schneider 2 cells, derived from a primary culture of late stage Drosophila melanogaster embryos.

“DPBS” is Dulbecco's phosphate buffered saline, a buffer having pH 7.2-7.6 at 25° C. and containing potassium chloride, potassium phosphate monobasic, sodium chloride, sodium phosphate dibasic, and optionally calcium chloride or magnesium chloride.

“EGS” is (ethylene glycol bis(succinimidyl succinate)), which is a crosslinking reagent that contains amine-reactive NHS-ester ends around a 12-atom spacer arm.

“Fluorophore” is a single fluorescent molecule that re-emits light specifically upon light excitation.

A “flow cell” is a specialized microscopic slide with multiple channels, used for the purpose of DNA sequencing.

“Formaldehyde” (“FA”) is a chemical having the formula CH₂O. It can be used as a chemical crosslinking reagent to crosslink DNA to protein or DNA to DNA within a chromatin complex to maintain chromatin structure.

“Genome” is the entire collection of DNA within an organism, including genes and non-coding regulatory regions.

“Genomic DNA” is the endogenous DNA within the chromatin of an organism.

“GM12878” is a human lymphoblastoid cell line.

“Hi-C” is an all-vs-all chromatin conformation capture method. The Hi-C method relies on PCR amplification to detect all genomic loci that interact in a genome.

“Klenow Fragment (3′→5′ exo-)” is an N-terminal truncation of DNA Polymerase I that is used for A-Tailing of free blunt DNA ends in chromatin complexes.

“LiCl” is lithium chloride.

A “linker” in the context of this disclosure is a short double stranded nucleic acid molecule capable of being bound a one end to chromatin DNA. A Linker can have a “T” overhanging nucleotide that serves as the substrate for “A” overhangs generated in the genomic DNA. For purposes of the application, the linker contains ligand, such as biotin, covalently linked on its 3′ terminal, and a fluorophore, such as the ALEXA 647 fluorophore, covalently linked on its 5′ terminal. The biotin facilitates the binding to the streptavidin-coated flow cell. Finally, the ALEXA 647 fluorophore reveals the presence of chromatin complexes.

“MES” is 2-(N-morpholino)ethansulfonic acid a buffer in the 6-8 pH range with a pKₐ of 6.10 at the 25° C.

An “Oligonucleotide” is a single stranded polynucleotide (˜30 nucleic acid bases).

“PAGE” is polyacrylamide gel electrophoresis, a method used to separate biological macromolecules, usually proteins or nucleic acids, according to their electrophoretic mobility.

“PBS” is Phosphate-Buffered Saline, a buffer solution used in biological research to simulate physiological conditions.

“PI” refers to protease inhibitors, which are molecules that inhibit the function of proteases (enzymes that digest proteins).

“RNAPII” is the RNA Polymerase II holoenzyme that is recruited to the promoters of protein-coding genes in living eukaryotic cells to catalyze the transcription of DNA to synthesize precursors of mRNA.

“SEQ LL” is a sequencing platform that performs single molecule sequencing called true single molecule sequencing (tSMS). Features of the SEQ LL (Woburn, Mass.) platform include streptavidin-coated flow cells and TIRF based imaging.

“Sequential sequencing” refers to multiple rounds of sequencing occurring sequentially on the same flow cell. Specifically, a unique DNA sequences, such as barcoded linkers, are first ligated onto the chromatin DNA. Complementary sequencing primers to the barcoded linkers are added to the flow cell one at a time, thereby allowing multiple rounds of DNA sequencing to be performed from the chromatin complex on the same flow cell.

“Sequencing primer” is a single stranded DNA oligonucleotide primer used in a sequential sequencing reaction. The sequencing primer is complementary to the template strand sequence present in the barcoded linkers, and primes the sequencing reaction.

“T4 DNA Ligase” is an enzyme that catalyzes the formation of a phosphodiester bond between juxtaposed 5′ phosphate and 3′ hydroxyl termini of double stranded nucleic acids.

“T4 DNA Polymerase” is an enzyme that catalyzes the synthesis of DNA in the 5′ to 3′ direction and requires the presence of template and primer. “TBST” is tris-buffered saline containing polysorbate 20 (TWEEN 20, Sigma-Aldrich). For example, TBST may contain 0.05 M Tris, 0.15 M NaCl, 0.1% TWEEN 20, pH 7.6 at 25° C.

“TCEP” is tris(2-carboxyethyl)phosphine, a reducing agent.

“TE” is a buffer containing Tris and EDTA.

“TIRF” refers to a Total Internal Reflection Fluorescence (TIRF) microscope. TIRF allows single molecule detection of fluorescently labeled molecules such as proteins or nucleic acids.

“TNE” is a buffer containing Tris-HCl, NaCl, and EDTA.

“USER” is Uracil Specific Excision Reagent, a mixture of Uracil DNA glycosylase (UDG) and the DNA glycosylase-lyase Endonuclease VIII that generates a single nucleotide gap at the location of a uracil. USER specifically removes the uracil nucleotides found in the non-template strand of the biotinylated linker, thus generating a single stranded region of DNA linker, allowing the sequencing primer to bind.

The present disclosure provides novel application of single molecule DNA sequencing to reveal functionally important 3D DNA spatial proximity.

### Assay Description

(I) Single Molecule Chromatin Interaction Assay (smChIA) Method

In one aspect, this disclosure provides smChIA, a method for determining contact interactions between loci in a single chromatin molecule.

The smChIA method has several advantages over earlier methods for assaying interactions between DNA loci in a chromatin complex. SmChIA can determine chromatin interactions between more than two chromatin loci, provide data from a single chromatin complex (a single molecule), and map this data to provide a view of chromatin interactions across a genome. The smChIA method can accomplish this without proximity ligation or DNA amplification. An extension of the smChIA method, single nucleus smChIA, can provide data on interactions within single chromatin complexes from a single nucleus. In the present smChIA method, many thousands of chromatin complexes (each made up of multiple molecules of DNA and protein) are generated by dual crosslinking using cross-linking agents such as formaldehyde (FA) and ethylene glycol bis(succinimidyl succinate (EGS). Chromatin complexes are then generated by sonication or restriction enzyme-based fragmentation. The fragmented chromatin complexes contain, for example, approximately 3 kb-8 kb DNA. Chromatin complexes are then enriched for complexes containing a protein of interest by ChIP. Free ends of DNA within the complexes are then prepared for ligating to a barcoded linker. For example, the free ends of DNA can be end blunted using T4 DNA polymerase, A-tailed using Klenow Fragment (3′-5′ exo-), and ligated on exposed ends with barcoded linkers. The barcoded linkers are unique DNA fragments containing a biotin molecule on each strand of their terminal end. One DNA strand of the double stranded barcoded linker can also contain a covalently bound fluorescent label. The chromatin complexes are then hybridized to a streptavidin-coated surface (e.g. a flow cell) to specifically bind the biotin containing DNA barcoded linkers. Finally, complexes are resolved by TIRF microscopy-based sequencing and protein imaging is carried out using fluorescent antibody immunostaining (e.g. on a SEQ LL platform). Genomic DNA sequence reads obtained from each complex with fixed physical location can then be mapped back to the reference genome to determine interactions between DNA loci in the genome and to determine which chromatin proteins are involved in these interactions.

The smChIA method has a number of important advantages over existing 3D genome mapping techniques such as ChIA-PET and Hi-C, both of which are population-based techniques. ChiA-PET and Hi-C detect interactions that occur with sufficient frequency to give rise to a signal. SmChIA is based on a single molecule protein detection and DNA sequencing platform and provides single molecule resolution. Detection is not statistically biased; less common interactions are detected as readily as frequent ones. In another embodiment the smChIA platform also permits simultaneous detection of histone modifications and genomic positions of individual nucleosomes. Histone modifications can be detected with the use of a fluorescently labelled antibody against a specific histone modification epitope.

Unlike ChiA-PET and Hi-C, smChIA does not use proximity ligation, a technique in which two free ends of DNA in a chromatin complex are ligated to each end of a DNA sequencing primer so that pairwise interactions between chromatin DNA fragments are detected. In smChIA, chromatin complexes are immobilized on a flow cell surface. The complexes are visualized by immunostaining, for example with transcription factor specific antibodies or histone protein specific antibodies. The barcoded linkers ligated to the free ends of DNA in the chromatin complex are used as primer binding sites. Single molecule sequencing using a different primer for each round of sequencing is conducted to produce sequential reads derived from the same optical spot on the flow cell surface. The sequences detected from these sequencing reads represent genomic loci involved in chromatin interactions.

The smChIA method also has the advantage of avoiding PCR amplification. Instead, individual chromatin complexes are analyzed. The smChIA method can serially detect multiple proteins in each chromatin complex. The method includes sequential rounds of single molecule sequencing of DNA fragments tethered together by proteins in the chromatin complex.

The smChIA technique can be used to create genome-wide single molecule chromatin interaction maps and also determine co-localization of multiple protein and RNA factors at each locus.

The present disclosure provides methods for preparing a chromatin sample from bulk cells for use in smChIA analysis.

SmChIA allows intact chromatin interaction complexes to be detected allowing single molecule resolution of DNA fragment in contact. The present method permits simultaneous detection of two or more DNA sequences physically associated with one another in 3D nuclear space as well as detection of the protein components of the complex.

SmChIA accomplishes detection of multiple interactions within a single chromatin complex in 4 main steps.

First, cross-linking cells to allow chromatin to remain intact, permeabilization of nuclei to allow in situ digestion. Alternatively, chromatin can be prepared using sonication.

Second, restriction enzyme digestion is used to generate chromatin complexes. To allow ligation of barcoded linkers, end blunting, and A-Tailing are performed.

Third, biotinylated and fluorescently labeled barcoded linkers are ligated genome wide.

Fourth, chromatin complexes are bound to a surface, such as a streptavidin-coated surface and imaged and sequenced using TIRF microscopy (e.g. the SEQ LL Platform). The chromatin complexes are sufficiently dispersed on the surface to permit resolution by TIRF microscopy. In certain embodiments the chromatin complexes should by at least 400 nm to permit resolution.

In addition to the steps for smChIA set forth in the SUMMARY section, the disclosure provides smChIA having the following characteristics.

The present disclosure provides a smChIA in which the first antibody, used for immunoprecipitation, and the second antibody, used for immunostaining are the same or different. In certain embodiments, the first and second antibody are the same.

The step of fragmenting the crosslinked genomic DNA in the smChIA can provide a plurality of chromatin complexes. Immunoprecipitation enriches the plurality of chromatin complexes for chromatin complexes containing the protein to which the first antibody binds. The enrichment for the chromatin complexes containing the protein to which the first antibody binds can by at least a factor of 2, a factor of 4, a factor of 10, a factor of 20, as compared to the plurality of chromatin complexes prior to the immunoprecipitation.

In certain embodiments the barcoded linker can contain a fluorescent label, such as an ALEXA FLUOR label, or any fluorescent label with an excitation and emission wavelength suitable for use with TIRF microscopy. In certain embodiments the barcoded linker is bound to a biotin molecule and the surface on which the chromatin complex is immobilized is a streptavidin-coated surface. The chromatin DNA can be subjected to end repair and A-Tailing prior to ligating the barcoded linker. In some embodiments at least 2, at least 4, or 2 to 8 different barcoded linkers are ligated to the genomic DNA in the chromatin complex. The barcoded linker may comprise barcoded an oligonucleotide selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In certain embodiments the barcoded linker comprises a template DNA strand comprising 10-100, 10-70, or 10-50 nucleotides covalently bound at the 3′-end to a biotin molecule and a non-template strand comprising uracil at multiple loci and fluorescent labeled at the 5′-end. The barcode itself is a short, unique sequence of 6-16 nt, 6-12 nt, or about 8-12 nt.

SmChIA may comprises the further step of de-crosslinking the barcoded chromatin complex after the barcoded chromatin complex is immobilized on the surface to release the proteins in the chromatin complex.

Crosslinking can be performed in the cell so as to allow the chromatin complex to remain intact in the cell and then can be followed by permeabilizing the cell. Crosslinking can be chemical crosslinking or UV crosslinking. Chemical crosslinking can be performed using suitable chemical crosslinking agents such as formaldehyde, methanol, or EGS or the like. In certain embodiments a combination of formaldehyde (0.5 to 3.0% v/v, or 1.0% v/v) and EGS (0.5 mM to 5.0 mM or 1.5 mM) is used for crosslinking.

The step of permeabilizing the cell can be performed mechanically, for example by sonication or by using a chemical membrane disrupter, such as a detergent, for example TRITON, TWEEN, NP40, SDS, or the like, or in particular instances SDS, for example 0.1% to 3.0% w/v SDS or 1.0% SDS may be used.

The fragmenting step can be performed by restriction enzyme digestion, such as MboI restriction enzyme digestion.

In another embodiment the disclosure provides a smChIA comprising the following steps:
a) crosslinking genomic DNA and proteins in a cell;
b) fragmenting the crosslinked genomic DNA to provide a chromatin complex, the chromatin complex containing DNA and one or more specific proteins;
c) ligating two or more different barcoded linkers to the DNA in the chromatin complex to form a barcoded chromatin complex;
d) immobilizing the barcoded chromatin complex onto a surface;
e) imaging the barcoded chromatin complex using a TIRF microscope at a single molecule level;
f) sequentially detecting proteins by immunostaining with a fluorescent labeled antibody against the specific protein present in the barcoded chromatin complex;
g) removing proteins from the chromatin complex, and retaining the DNA templates of the barcoded chromatin complex that are immobilized on surface;
h) sequentially sequencing the DNA in the barcoded chromatin complex at a single molecule level to generate a plurality of sequence reads; and
i) mapping said plurality of sequence reads to a referenced genome to produce a genomic location of said sequenced reads to generate a 3D genomic connectivity map, wherein said connectivity map is indicative a physical interaction between the genomic DNA and proteins present in the chromatin complex at a single molecule level.
   (II) Single Nucleus Single Molecule Chromatin Interaction Assay (Single Nucleus smChIA)

In another aspect, the present disclosure provides a single nucleus smChIA. A method for preparing a chromatin sample from a single nucleus on a solid surface for smChIA analysis is disclosed. The single nucleus smChIA is an extension of the smChIA protocol developed for bulk cellular samples, but the resolution and analysis are conducted at the single nucleus level.

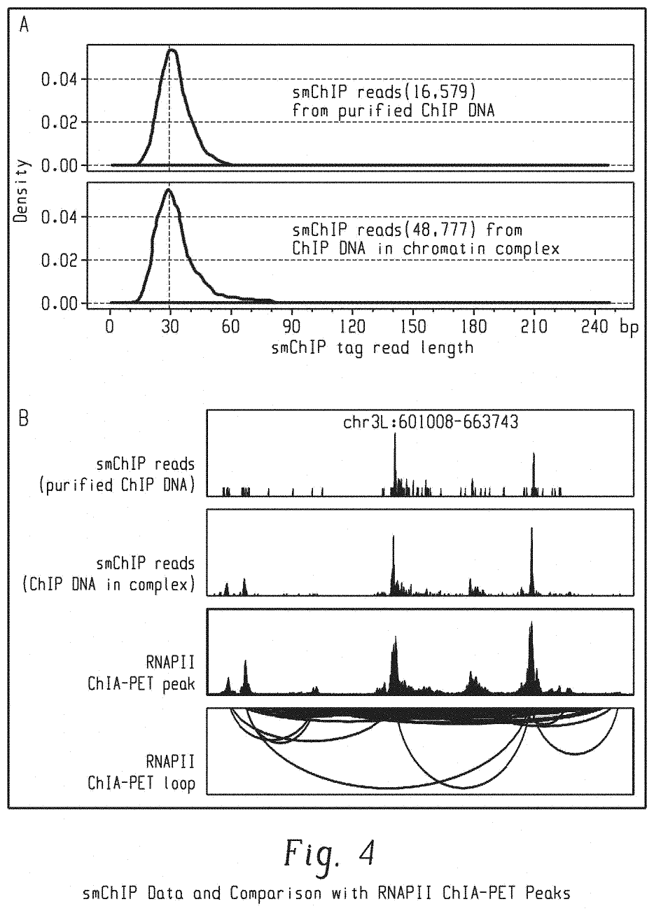

In the single nucleus smChIA, purified nuclei are subjected to in situ chromatin digestion by a restriction enzyme, followed by ligation with barcoded linkers. Each barcoded linker is biotin labeled and also labeled with a fluorescent marker. Nuclei are loaded onto a surface, such as a streptavidin-coated surface (e.g. a flow cell) in dilution, so that the nuclei are sparsely positioned on the surface in layer of a buffer solution, e.g. a PBS solution. Then, the nuclei are permeabilized on the streptavidin-coated surface, under gentle lysis conditions, such as 0.5% SDS for 15 minutes. Nuclei may be permeabilized by any suitable detergent such as SDS, TRITON, such as TRITON X-100, digitonin, saponin, or TWEEN such as TWEEN-20, or NP40, or with the use of enzymes such as proteinase K and streptolysin O or the like. The biotin-labeled chromatin complexes are released from the permeabilized nuclei, dispersed radially, and immobilized within a defined micro-region on the glass surface. The effect is to separate the chromatin complexes sufficiently for smChIA but affix the complexes from each nucleus within a small area so that they may easily be identified as having come from the same nucleus. Next, the chromatin complexes immobilized on the flow cell surface are subjected to smChIA as described above (FIG. 4A). There are two key issues for application of the smChIA method to a single nucleus. First, biotin-labeled, barcoded linkers need to be ligated with fragmented chromatin DNA in situ. Second, single nuclei must be dispersed enough for smChIA sequencing while remaining close enough to their origin to distinguish individual nuclei.

In addition to the step for single nucleus smChIA set forth in the SUMMARY section the disclosure also provides a single nucleus smChIA having the following characteristics. In certain embodiments the crosslinking step may be performed by any of the means described above for smChIA. In certain embodiments the barcoded linker may have any of the limitations described above for barcoded linkers used for smChIA. The step of fragmenting the chromatin DNA to produce chromatin complexes may also be performed by any of the methods described for smChIA. Like smChIA, single nuclear smChIA can include immunostaining. The antibody used in the immunostaining step can be any of the antibodies described for immunostaining for smChIA. As in smChIA the chromatin complexes in single nucleus smChIA may be immobilized on a streptavidin-coated surface.

The nuclear lysing step may be performed using a detergent, such as TWEEN, TRITON, NP40, SDS, or the like, or in certain instances SDS, for example 0.1% to 3.0% w/v SDS, or 0.5% w/v SDS, or by ExM, described below can be used.

In another embodiment the disclosure provides a single nuclei smChIA comprising the following steps:
a) providing a single nucleus, said nucleus comprising genomic DNA and proteins;
b) crosslinking genomic DNA and proteins in the nucleus;
c) fragmenting the crosslinked genomic DNA in situ to provide a plurality of chromatin complexes, each chromatin complex containing genomic DNA and one or more specific proteins;
d) ligating two or more different barcoded linkers to the DNA in the chromatin complexes, to form barcoded chromatin complexes;
e) immobilizing said single nucleus onto a surface;
f) lysing said single nucleus such that the barcoded chromatin complexes contained in the nucleus are dispersed on the surface;
g) immunostaining the barcoded chromatin complex with an antibody capable of binding to the specific protein present in the barcoded chromatin complex;
h) imaging the immunostained barcoded chromatin complex using a TIRF microscope;
i) repeating the steps g) and h) for sequential detection for two or more antibodies;
j) sequential sequencing of the barcoded chromatin complex at a single molecule level to generate a plurality of sequence reads; and
l) mapping the plurality of sequence reads to a referenced genome to produce a genomic location of the sequenced reads to generate a 3D genomic connectivity map, wherein said connectivity map is indicative a physical interaction between genomic DNA and proteins in the chromatin complex at a single molecule level.
   Alternate Method for smChIA Applied to Single Nuclei (SN)/Single Cell (SC)

In yet another aspect, the present disclosure provides an alternate method of single-nucleus chromatin preparation by provides a means of reliably dispersing the chromatin complexes onto the flow cell surface sufficiently to separate each chromatin complex unit and yet localize the chromatin complexes from a nucleus sufficiently close to each other to retain the nuclear boundary. This method uses expansion microcopy (ExM) to lyse the nuclei (Chen, F., et al., Science (2015) 347(6221): 543-548; DOI: 10.1126/science.1260088). ExM uses an expandable (swellable) polyelectrolyte gel matrix to fix cellular materials, including proteins and nucleotides, in the cell, followed by osmotic swelling of the cell-gel composite. ExM preserves the original cell structure. It has been applied to tissue slices and single cells for detecting proteins and RNA at super resolution using conventional microscopy (Chen, F. et al., Nature Methods (2016) 13: 679-684; DOI:10.1038/NMETH.3899). Recent advances of this method, called iterative expansion microscopy (iExM) have been used to expand samples up to 22-fold (Chang, J-B., et al., Nature Methods (2017) 14: 593-599; DOI:10.1038/nmeth.4261).

iExM is used to infuse nuclei (in situ digested and DNA oligo ligated) with polyelectrolyte gel, and thus chemically anchor all the digested chromatin complexes in relative positions in situ. The osmotic conditions are altered to expand the nucleus-gel composite to separate each individual chromatin complex by a distance of approximate 20-fold. A cover slide is applied to press the expanded nuclei (3-dimension) downward on the flow cell surface (2-dimension). The chromatin complexes are then immobilized onto the flow cell surface and using a ligand/substrate system such as biotin/streptavidin. In certain embodiments, biotin can be covalently bound to the barcoded linker bound to the chromatin DNA and streptavidin can be incorporated onto the flow cell surface. Integration of iExM into single cell smChIA protocol will facilitate the preservation of the native nuclear structure and sufficiently expand distinct chromatin complexes for single molecule protein detection and DNA sequencing. Considering the average human nucleus is 5-10 mm³, iExM expansion will enlarge the nuclei to 200 mm³ in 3 dimensions, and possibly 400 mm² after pressing into 2 dimensions onto the flowcell surface. A regular microscopic glass slide (75 mm×25 mm) will contain at least 5,000 nuclei for smChIA analysis.

### Cell Harvesting and Crosslinking

About 1×10⁸ cells can be single or dual cross linked using 1% formaldehyde and 1.5 mM EGS. Cross-linked cells may be stored at −80° C.

### Cell Lysis

To carry out cell lysis, cells are washed with a buffer, such as PBS (with PI), twice at room temperature. Cell lysis and nuclear lysis can be carried out in 10 ml of 0.1% Cell Lysis Buffer (50 mM Tris pH 7.5, 150 mM NaCl, 1 mM EDTA) without TRITON X-100 at room temperature (RT) for 6 min. 900 μl of 10% SDS at 37° C. are then added, the cells are rotated on INTELLI-MIXER (Elmi Ltd., Riga, Latvia) at 10 rpm for 10 min. Lysis is determined by visual observation under microscope for hallmark visual indicators of lysis. If the cells are not lysed well, the process can be repeated. The cells are then twice washed with 0.1% Cell Lysis Buffer with proteinase inhibitor (PI), suspended in 10 ml ice-cold 0.1% Cell Lysis Buffer with TRITON X-100 (PI) for sonication. TRITON is added to prevent chromatin precipitation when sample is at 4° C.

### Chromatin Complex Generation

Chromatin DNA can be sheared into approximately 3 kb, or fragments containing at least 3 kb DNA, at least 5 kb DNA, 3 kb-10 kb DNA, 5 kb-10 kb DNA, approximately 8 kb DNA, or 8-10 kb DNA. Chromatin can be sheared with sonication, restriction enzyme digestion, or other suitable methods. Suitable enzymes for restriction enzyme digestion include Mbo I and Pvu II. If sonication is used, aliquot chromatin DNA to tubes for sonication, for example using a Branson Digital Sonifer Cell Disrupter at 38% amplitude, 20 sec on/30 sec off, for 6 min. Samples are kept cold to avoid overheating. Chromatin DNA is then centrifuged for at least 5 min at 1700×g.

### ChIP Enrichment of Complexes

The chromatin complexes may be obtained through Chromatin immunoprecipitation (ChIP).

ChIP can be used to enrich samples of chromatin complexes for the complexes that contain a specific protein of interest. Having a sample of chromatin complexes enriched for a particular protein allows the identification of genomic regions associated with that proteins such as histones and other proteins binding to nucleic acids in nucleic-acid protein complexes (reviewed in Taverner et al., Genome Biol, 2004. 5(3): p. 210). In ChIP, proteins are cross-linked with DNA at their sites of interaction. Crosslinking can be accomplished quickly and efficiently by adding a suitable fixative such as formaldehyde, glutaldehyde, EGS, or methanol directly to living cells in culture.

Crude extracts of these fixed cells are then prepared, and the chromatin sheared by sonication, hydroshearing, repeated drawing through a hypodermic syringe needle or by restriction enzyme digestion to an average size of usually about 1 kb, then used in immunoprecipitation reactions with antibodies raised against the DNA-associated protein of interest (e.g. transcription factors or histones). DNA fragments enriched in each immunoprecipitation are then de-linked and purified to allow their identification by a variety of methods. An advantage of using ChIP is that this approach is able to “freeze” the in vivo gene regulatory network by rapid crosslinking of chromatin and other non-histone proteins, thereby providing a picture of the regulatory system at any point in time, free of potential artifacts imposed by heterologous expression, for instance.

To prepare chromatin complexes containing a protein of interest (for example, RNAPII), protein G magnetic beads can be coated in an RNAPII antibody by incubation and rotation at 4° C.

Antibody coating incubation can be done up to 24 hours ahead of time, but should be done for at least 6 hours. RNAPII antibody is incubated with protein G beads as follows. First, 1 ml of protein G magnetic beads are washed with PBS/0.1% TRITON X-100 twice, beads are suspended with 7 ml of PBS/0.1% TRITON X-100 and incubated with the antibody of interest at 4° C. and rotated at 12 rpm about 6-8 hours. The protein beads can be DYNABEADS Protein G for Immunoprecipitation from Thermo Fisher Scientific, Waltham, Mass., catalog no. 10003D.

Chromatin can be prepared from cells of interest, for example GM12878 cells or Drosophilia S2 cells. In an example using GM12878 cells, 1×10⁸ GM12878 cells are washed with room temperature PBS (PI) twice.

To effect cell and nuclear lysis, GM12878 cells are suspended in 10 ml of 0.1% Cell Lysis Buffer without with proteinase inhibitor at room temperature for 6 minutes. 900 μl of 10% SDS at 37° C. are then added and the cells are rotated at 10 rpm for 10 minutes. The cells are viewed under microscope to determine whether lysis has occurred. If cells are not sufficiently lysed, the lysis procedure is repeated. Cells are then twice washed with 0.1% Cell Lysis Buffer (no TRITON, PI), suspended in 10 ml ice-cold 10 ml 0.1% Cell Lysis Buffer with TRITON X-100 (PI) for sonication. Cells are aliquoted to tubes for sonication. Cells are sonicated using a Branson Digital Sonifer Cell Disrupter at 38% amplitude, 20 seconds on/30 seconds off for 6 minutes, and then spun down for 5 minutes at 1,700 g.

Chromatin can be pre-cleared to remove background binding of chromatin to beads. This can be done by incubating 1 ml of protein G magnetic beads with the sonicated chromatin complexes, at 4° C. on a rotor for at least 2 hours. After incubation, the supernatant contains the precleared chromatin, and can be transferred to a new tube. The RNAPII antibody bound beads can be washed with 0.1% triton/PBS three times to remove non-bound antibody.

To set up the ChIP, discard the supernatant of antibody bound beads, transfer the precleared chromatin complex supernatant into the tube with antibody bound beads, and incubate overnight at 4° C. while rotating. ChIP wash steps then are performed to remove non-specific binding of complexes. 20 μl of chromatin are reserved for fragment size quality control. ChIP wash step includes a high salt buffer wash such as once with 0.1% SDS/.35M NaCl (PI) Cell Lysis Buffer, one wash with LiCl buffer, followed by TE (PI) buffer wash.

### Preparation and Ligation of Barcoded Linkers

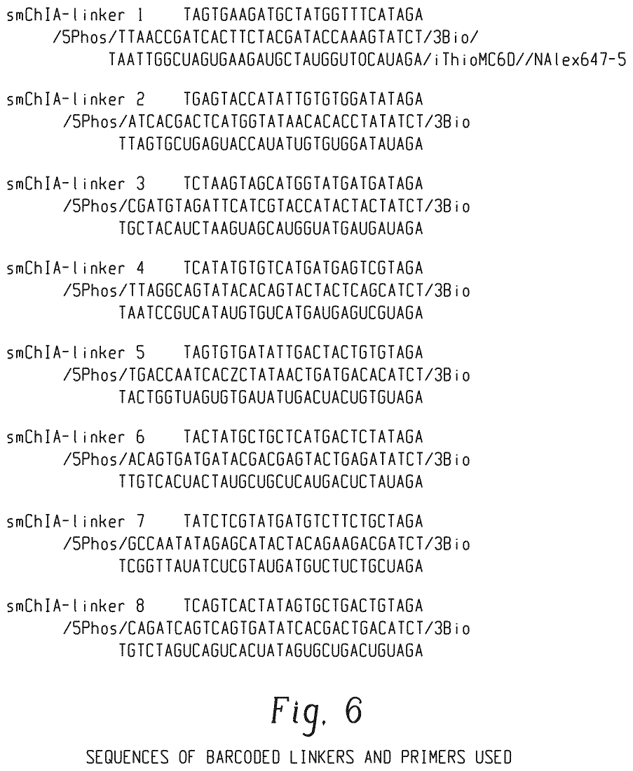

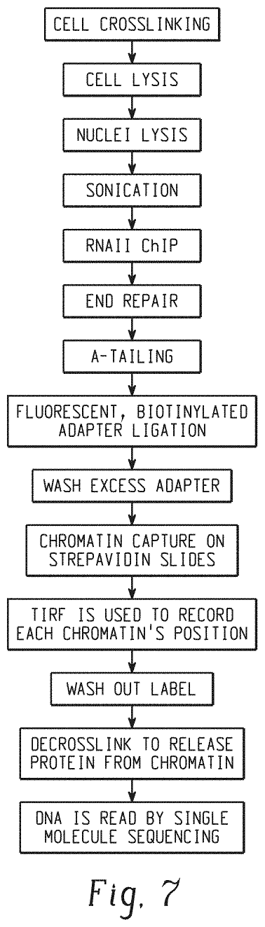

SmChIA barcoded linkers (oligonucleotides) such as those shown in FIG. 6 are synthesized. Biotin modification is covalently bound to the 3′ end of the template strand. Thymine is replaced by uracil at multiple locations in the non-template strand, the non-template strand is fluorescent labeled, for example with ALEXA FLUOR 647, or other suitable fluorescent label for DNA sequencing. In addition to ALEXA FLUOR 647 any fluorescent dye with a emission wavelength detectable by TIRF microscopy can be used, for example having an absorption wavelength of about 350 nm to about 740 nm, and an emission wavelength about 15 to 50 nm longer than the absorption wavelength, or about 350 nm to about 370 nm to about 770 nM. The fluorescent label detectable by TIRF may have an absorption wavelength of about 480 nm to about 680 nm and an emission wavelength about 15 to 50 nm longer than the absorption wavelength. In addition to ALEXA FLOUR 647 other fluorescent labels that may be used include ALEXA FLUOR 488, 532, 546, 568, and 594, CY2, CY3, CY3B, CY5, CY5.5, CY7, DYLIGHT488, 550, 594, 633, and 650 (Thermo Fisher Scientific), ATTO 488, 532, 565, 590, 647N, and 680. ATTO RHO3B, ATTO RHO11, ATTO RHO3B, and Rhodamine 6G.

Biotinylated barcoded adapters, once designed, may be obtained from a commercial source such as Integrated DNA Technologies (www.idtdna.com).

Single stranded smChIA barcoded linker oligos are dissolved in 1×THE buffer and allowed to incubate at 4° C. overnight to prepare double stranded adaptor. The strands of the smChIA barcoded linker is annealed form a double to form a double stranded barcoded linker and run PAGE for quality control. The smChIA barcoded linker is diluted to 200 ng/μl before use in the following experiments.

Genomic DNA fragment ends located within the antibody enriched chromatin complexes need to be repaired prior to ligation of barcoded linker. The linker ligation can be designed as blunt end ligation or sticky end ligation. In this example, sticky end ligation is used. First, end repair is performed to blunt all ends of genomic DNA. A-Tailing of the now blunted 3′ end of genomic DNA fragments follows the end repair step. The linker contains a “T” overhang that will bind complementary to the genomic DNA fragment ends. Biotinylated barcoded linkers (for example having 10-50 bp, 20-40 bp, 30-35 bp, or 33 bp) can be generated by hybridization of single stranded oligonucleotides. Linkers can be ligated to genomic DNA by a complementary T/A overhang-based ligation. A mixture of 2, more than 2, 3-20, 4-10, 4-8, 6-8, or 8 barcoded linkers is ligated to the chromatin complexes to generate chromatin complexes containing multiple distinct barcoded linkers to allow multiple sequence reads to be obtained from each chromatin complex.

### Chromatin Loading to Flow Cell

Chromatin complexes are loaded and specifically bind to the streptavidin-coated flow cell by their biotinylated linkers. The concentration of the chromatin complexes must be determined experimentally and will be dependent on the DNA fragment distribution of the library as well as the proteins content of the sample.

### TIRF Based Protein Imaging

Imaging of the protein component of chromatin complexes can be carried out using a dot-blot style assay. After chromatin complexes are loaded, the flow cell is blocked with 4 ml of blocking buffer (TBST containing 5% non-fat dried milk) for 4 hours to decrease non-specific binding of the fluorescent antibody. Next, the flow cell is washed 3 times with TBST, and primary antibody is added. The primary antibody can be incubated overnight at 4° C. on a rotor. The array is washed 3 times in TBST to remove excess primary antibody. The second antibody is added to the flow cell and left to incubate for 1 hour at room temperature. The flow cell is then washed again 3 times in TBST to remove unbound secondary antibody, and signal is detected by FLUORCHEM Q (Protein Simple, San Jose, Calif.). Donkey Anti-Mouse IgG H&L (ALEXA FLUOR 647, ThermoFisher Scientific) pre-adsorbed, is one suitable second antibody detection system.

Antibodies are diluted in imaging buffer to a final concentration of 50-100 ng/ml, and images are taken every 15 min. for a total incubation time of 3 hours. For experiments requiring imaging of multiple protein targets the flow cell is washed extensively with imaging buffer (10 washes×5 min incubation for each wash). All positions are imaged again and residual spots excluded from further analysis. Additional antibodies can then be applied and imaged as described for the first antibody.

### Sequential DNA Sequencing

Single molecule scripts are adapted to disable fluidics while imaging the flow cell for antibody binding and dissociation events over time.

De-crosslinking can be done to release protein from the chromatin complex. The flow cell can be washed with 2M NaCl for 10 min, and increase temperature to 37° C. The de-crosslinking process may be performed with the following two methods.

Method 1: Incubated with proteinase K in TE buffer contained 0.5% SDS buffer at 37° C.-65° C. overnight or at least 4 hours.

Method 2: Incubate with TE buffer containing 0.5% SDS buffer for 65° C. for 2 hours, then incubate with proteinase K in TE buffer containing 0.5% SDS buffer for 4 hours or overnight.

USER restriction enzyme is applied, and allowed to incubate for 1 hour at 37° C. on the flow cell to remove uracil nucleotides within one strand of the linker, allowing sequencing primers to access and hybridize to the template strand.

The flow cell can then be washed several times with H₂O and pre-heated to 55° C. to prepare the sequencing reaction. Sequential DNA sequencing is carried out using one primer at a time, added to the flow cell at a final concentration of 10 nM. The sequencing primer is allowed to hybridize for 20 minutes, followed by blocking to quench the sequencing reaction and washing to remove unbound primer. Single molecule sequencing is carried out through multiple rounds of sequencing using unique barcoded primers each time to specifically sequence one barcoded DNA template at a time.

### Reference Genome Mapping

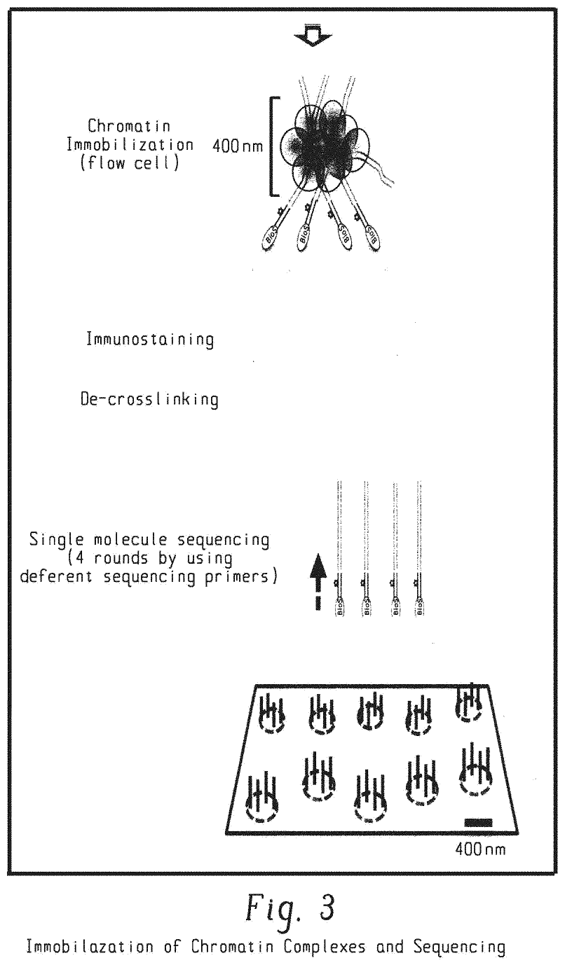

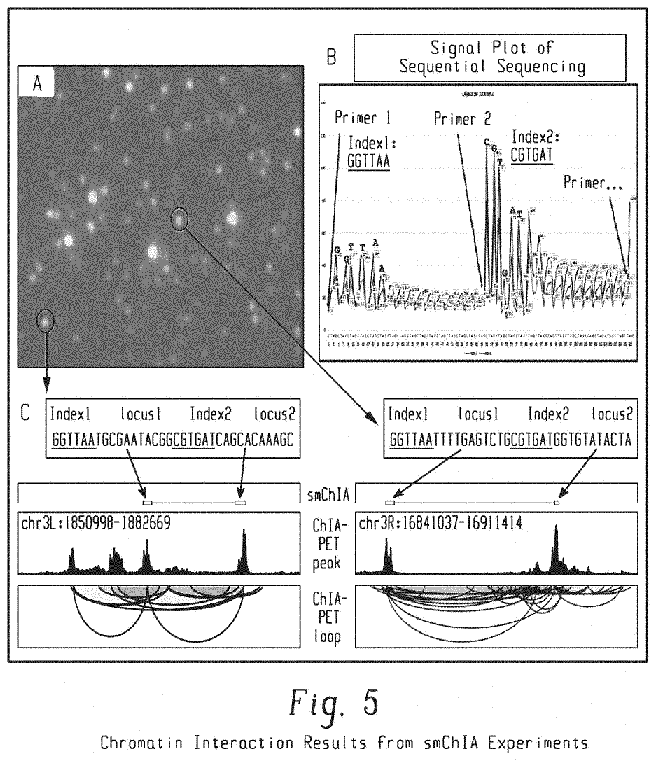

The immobilized DNA fragments on the substrate surface are sequenced using one primer (P1). In each sequencing run multiple fragment sequences (reads) are generated for the P1 primer. Most of the reads have been found to be about 30 bp, but can be 10-15 bp, 20-100 bp, 30-80 bp, or 40 to 50 bp (FIG. 4A). These reads are mapped to a reference genome. A Drosophila reference genome (dm3) was used for the map shown in FIG. 4B. In one aspect, this disclosure provides a method for identifying chromatin interaction events mediated by specific DNA binding proteins, such as histones, across long distances and between different chromosomes. In another aspect, the disclosure provides an isolated oligonucleotide comprising at least one first tag and at least one second tag, wherein the first tag is obtained from a first polynucleotide and the second tag obtained from a second polynucleotide, the first and second polynucleotides obtained from a nucleic acid-protein complex. The tags correspond to regions of chromatin in nucleic acid-protein complexes. These tags may then be sequenced to analyze, identify, and/or detect chromatin interaction events (FIGS. 3 and 5).

### Linker

The linker may be any DNA oligonucleotide. The linker may contain a peptide or other molecule capable of selective binding to the substrate, for example the linker may contain biotin, which binds to a streptavidin- or avidin-coated substrate. The linker also contains a fluorescent label. FIG. 6 shows 8 barcoded linkers, the attachment of biotin to the linkers and the primers specific to these linker, which may be used in the smChIA method. The 5′-3′ sequence for the barcoded linkers, which biotinylated at the 3′ end are provided as SEQ ID NOS: 1-8, the sequences for the complementary strands, shown 3′-5′ in FIG. 6 and labeled at the 5′ end with the ALEXA FLUOR 647 fluorophore are provided as SEQ ID NOS: 9-16. These sequences incorporate uracil in place of thymine at multiple loci. Primers sequences to the barcoded linkes are provided as SEQ ID NOS: 17-24.

Development of smChIA System

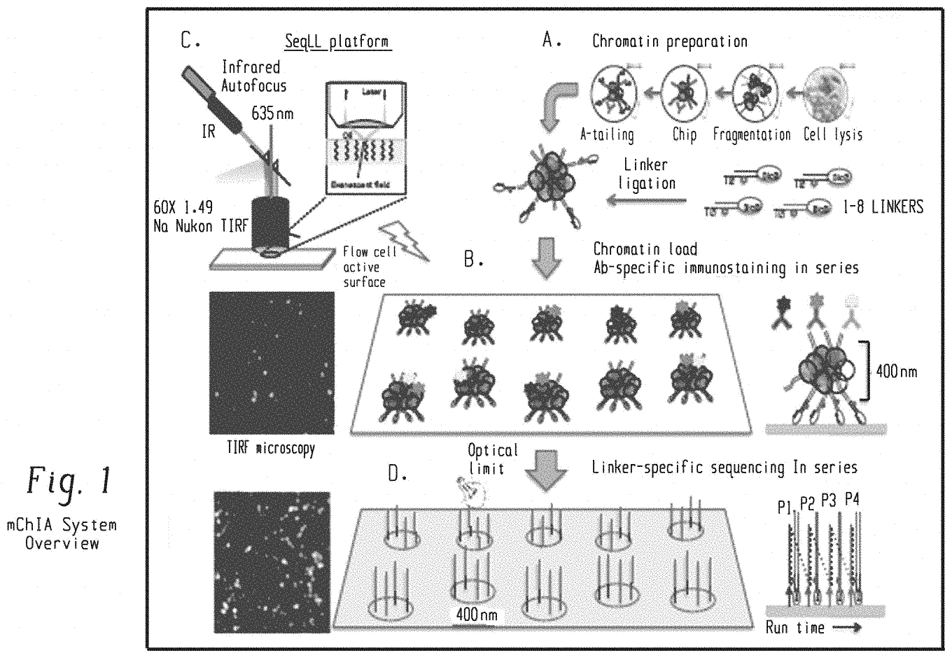

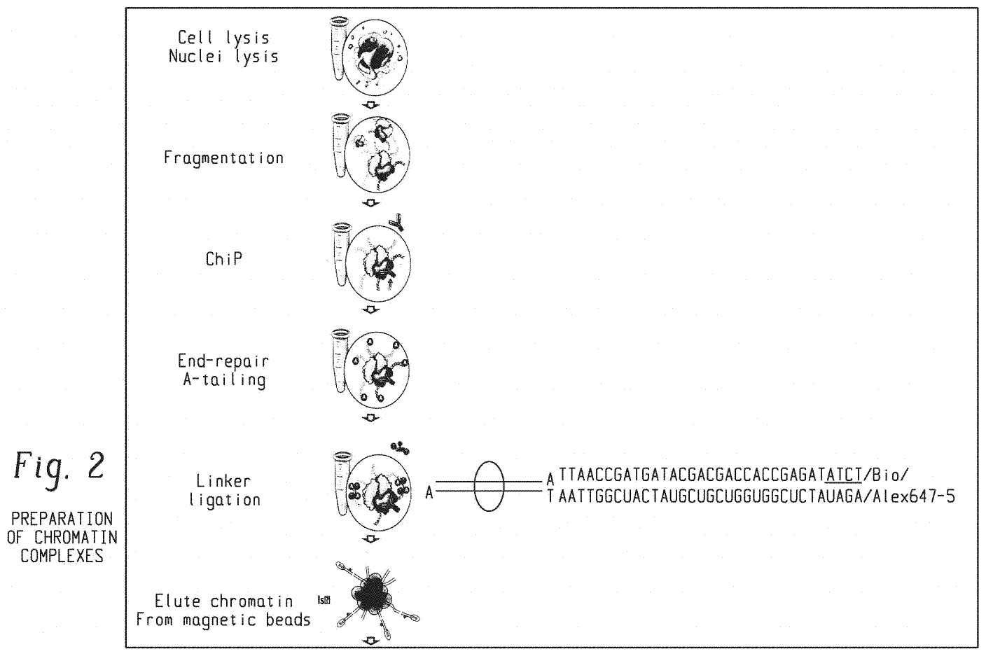

We estimated that each chromatin complex might tether up to 8 DNA fragments representing multiple genomic loci involved in a chromatin interaction with an a chromatin protein, such as RNAPII. The size of each DNA cluster is approximately 400 nm in diameter, and 400 nm is the optical resolution limit of TIRF microscopy, presenting a technical challenge of how to distinguish the sequencing reads derived from different DNA templates within each cluster. The technical problem of distinguishing sequencing reads from discrete DNA templates within a cluster was overcome with a sequential sequencing strategy (FIG. 1). This sequencing strategy includes the use of barcoded DNA linkers. The multiple DNA fragments in each chromatin complex were randomly subject to ligation to each of the distinctive barcoded linkers.

Once the DNA fragments, labeled with their barcoded linkers are immobilized, the DNA fragments can be discretely sequenced using linker-specific primers one at a time in series. Such sequential readouts of the DNA sequences in each cluster (optical spot detected by TIRF) are mapped to the reference genome and found to reflect chromatin interactions involving multiple loci mediated by the proteins identified by immunostaining, which can be performed before or after sequencing.

FIG. 1 depicts the development of smChIA system and its multiple steps.

(i) Chromatin Preparation:

FIG. 1A depicts the process of chromatin preparation. Chromatin samples are prepared through cell/nucleus lysis, fragmentation, ChIP, and end repair plus A-Tailing, followed by DNA linker ligation. Each linker can contain dsDNA with a T overhang at the 3′ end, a biotin group, a unique DNA sequence barcode, multiple uracil (U) bases distributed within the non-template strand, and a fluorescent label. Typically, at least two and up to eight different barcoded linkers are ligated to the chromatin samples.

(ii) Chromatin Loading and Antibody (Ab)-Specific Immunostaining in Series:

FIG. 1B depicts chromatin loading onto the flow cell. Chromatin samples are loaded to streptavidin-coated flow cell surface, and each chromatin complex is immobilized through biotin-streptavidin conjugation. The chromatin complexes are sufficiently diluted so that there is a >1 μm distance between adjacent complexes. Proteins in the chromatin complexes are then visualized by serial immunostaining directly on the flow cell with antibodies specific to the proteins of interest.

In certain embodiments, the immunostaining procedure is repeated several times to detect a number different chromatin-bound proteins, including general transcription factors (TFs) such as RNAPII (RNA polymerase II), specific TFs such as RARA (retinoic acid receptor), ER (estrogen receptor), etc., and chromatin architecture factors such as CTCF (CTCC-binding factor), Cohesin, etc. Any chromatin protein for which a ChIP grade antibody is available may be detected. Additional chromatin-bound proteins that may be immunostained by this procedure include chromatin proteins which bind to the following ChIP grade antibodies which can be found at https://www.diagenode.com/en/categories/chip-grade-antibodies: H3R2me2, AF9, AML1-ETO, BRD4, C/EBP, CBFb, CBX2, CBX8, CHD1, CHD7, CRISPR/Cas9, CTCF, CXXC1, DNMT3B, E2F6, ERR, ETO, EZH2, FOXA1, FOXA2, FOXM1, FUBP1, GR, GTF2E2, histone H2A.X, H2A.Z, H2A.Zac, H2A.ZK4ac, H2A.ZK7ac, H2AK119ub, H2AK5ac, H2BK12ac, H2BK15ac, H2BK20ac, H2BK123ub, H2Bpan, H3.3, H3K14ac, H3K18ac, H3K18me1, H3K18me2, H3K23me2, H3K27ac, H3K27me1, H3K27me2, H3K27me3, H3K27me3S28p, H3K36me1, H3K36me2, H3K36me3, H3K4ac, H3K4me1, H3K4me2, H3K4me3, H3K4me3T6p, H3k4un, H3K56ac, H3K56me1, H3K64me3, H3K79ac, H3K79me1, H3K79me3, H3K9/14ac, H3K9ac, H3K9acS10p, H3K9me1, H3K9me2, H3K9me3, H3Kme3S10p, H3K9un, H3pan, H3R17me2, H3R17me2(asym), H3R17me2(asym)K18ac, H3R2me2K4me2, H3T6pK9me3, H4K12ac, H4K 16ac, H4K20ac, H4K20me1, H4K20me2, H4K20me3, H4K5,8,12ac, H4K5ac, H4K8ac, H4pan, H4S1p, HDAC1, HDAC2, HDAC3, HIF1alpha, HP1, JARID1C, JMJ2a, JMJD6, KAP1, KAT2B, KDM6A, LSD1, MBD1, MBD1, MeCP2, MYH11, NCOR1, NF-E2, NFKB, NFYB, NRF 1, NRF2, OCT4, p300, p53, PARP1, PAX8, Pol II, Pol II S2p, PPARG, RbAp48, RBBP5, RFX-AP, RNF2, SAP30, SIN3A, Ski3, Ski8, SMAD1, SMAD2, SMYD3, Suz12, TAL1, TARDBP, TRP, TFIIF, THOC1, TIP5, TRRAP, Ty1, UHRF1, YY1, ZHX2, and ZMYM3. Serial immunostaining allows the identification of individual and multiple co-localized proteins at each chromatin complex. Immunostaining of proteins of interest can be performed before or after the sequential sequencing step.

The protein component of the chromatin complex can be removed via reverse-crosslinking prior to sequencing. De-crossslinking leads to the purification of the DNA component of the chromatin complexes, which was previously tethered together by the chromatin proteins. The genomic DNA remained on the flow cell surface tethered by the terminal biotin group contained in the barcoded linker, as distinct clusters of DNA fragments. Within the individual clusters, each of DNA fragment represents an individual genomic locus, and the different DNA fragments within each cluster represent multiple genomic loci that were brought together through long-range chromatin interactions mediated by chromatin proteins.

(iii) SEQ LL Platform:

FIG. 1C depicts the SEQ LL platform, which uses Total Internal Reflection Fluorescence (TIRF) microscopy for single molecule protein detection and DNA sequencing. The SEQ LL platform does not require PCR amplification. FIG. 1D depicts DNA fragments tethered in discrete chromatin complexes. The discrete chromatin complexes are ˜400 nm in diameter, the optical resolution limit of TIRF. The discrete chromatin complexes are adequately dispersed on the flow cell surface so as not to overlap. To read multiple DNA fragments from each of the DNA clustered in a single chromatin complex, a sequential sequencing strategy is implemented in which specific primers (P1, P2, P3, etc.) corresponding to each of the DNA linkers are applied in series to allow multiple DNA fragments from a single chromatin complex to be sequenced, facilitating single molecule sequencing. Each dotted line indicates a sequential linker-specific sequencing run.

### Use of Drosophila S2 Cells

Drosophila S2 cells were used in the development of smChIA. Drosophila have a relatively small genome, and there is already an abundance of 3D genome organization data (Hi-C and ChIA-PET) for Drosophila S2 cells, permitting facile comparison of preliminary results. RNAPII-mediated chromatin interactions were studied and existing RNAPII ChIA-PET data were compared with the collected smChIA data for technical validation.

Immunostaining for RNAPII on chromatin complexes immobilized on the streptavidin-coated glass surface, provided excellent results comparable to those for histone modification markers to individual nucleosomes using the SEQ LL system (Shema, E., et al., Science (2016) 352(6286): 717-721, PMID:27151869).

### EXAMPLES

The following examples are provided to further illustrate various preferred embodiments and techniques of the invention. It should be understood, however, that these examples do not limit the scope of the invention described in the claims. Many variations and modifications are intended to be within the spirit and scope of the invention.

### Example 1. smChIA Barcoded Linker Preparation

smChIA barcoded linker oligonucleotides (such as the sequences shown in FIG. 6) are synthesized. The 3′ end of the template strand is biotinylated. The non-template strand has thymine replaced by uracil (U) at multiple loci. The non-template strand is fluorescent labeled, for example with ALEXA FLUOR 647. Barcoded linkers used in the following experiments were synthesized by Integrated DNA Technologies, IDT (https://www.idtdna.com/).

The dry single stranded smChIA barcoded linker oligos are dissolved in 1×THE buffer and incubated at 4° C. overnight to prepare double stranded barcoded linker. The barcoded linker strands can be annealed in a thermal cycler by ramping from 95° C. to 20° C. gradually over 10 minutes and run on PAGE for quality control. The smChIA barcoded adaptor is diluted to 200 ng/μl for the following experiments.

### Example 2. Generation of Chromatin Complexes from GM12878 Cells Enriched for RNAP II by ChIP

GM12878 or Drosophila S2 cells were single or dual crosslinked with EGS and 1% FA and stored at −80° C. until needed.

RNAPII antibody bounded to protein G beads by twice washing 1 ml of protein G beads with PBS/0.1% TRITON-100 twice. The beads are suspended with 7 ml of PBS/0.1% TRITON X-100 and incubated with rotation at 4° C. for 6-8 hours.

1×10⁸ GM12878 cells are washed with room temperature PBS (PI) twice. Cell and nuclear lysis is effected by adding cells to 10 ml of 0.1% Cell Lysis Buffer (PI) at room temperature for 6 minutes. 900 μl of 10% SDS are then added at 37° C. and cells are rotated at 10 rpm for 10 minutes. Cells are viewed under a microscope. If lysis is not sufficiently complete the lysis procedure is repeated. Once lysis is sufficiently complete, cells are washed with 0.1% Cell Lysis Buffer (no TRITON or PI) twice and suspended in 10 ml ice-cold 0.1% Cell Lysis Buffer with TRITON X-100 (PI) for sonication.

Cells are aliquoted to tubes for sonication. Cells are sonicated at 38% amplitude for 20 seconds on/30 seconds off 6 minutes, and then centrifuged for 5 minutes at 1700 g.

Chromatin are precleared by incubating 1 ml of protein G magnetic beads with the sonicated chromatin complexes, at 4° C. with rotation for at least 2 hours. The sonicated chromatin and protein G magnetic beads are centrifuged at low speed (˜100×g) for 1 minute at 4° C. The supernatant contains the precleared chromatin.

Excess RNAPII is removed from the protein G magnetic beads by washing RNAPII bounded antibody beads with 0.1% triton/PBS three times.

To perform ChIP the supernatant of the antibody bound beads is discarded. The pre-cleared chromatin are transferred to the antibody bound beads and incubated overnight at 4° C. A 20 μl aliquot is reserved of chromatin for fragment size quality control.

Following overnight incubation of the chromatin and RNAP II antibody bound beads, the beads are washed with 0.1% Cell Lysis Buffer (PI) three times; 0.1% Cell Lysis Buffer/350 mM NaCl (PI) once, LiCl buffer once, and TE (PI) three times.

### Example 3. End-Blunting the Sonicated DNA Fragment

The chromatin bound to the antibody-bound beads is washed with wash buffer and then washed with ice cold TE Buffer (Ambion, AM9849, nuclease free).

T4 Polymerase master mix (at 1.2 times desired volume) is prepared in a new tube, on ice. The master mix requires 615.8 μl nuclease-free water, 170 μl 0× Buffer for T4 DNA polymerase, and 170 μl 10 mM dNTPs.

The TE buffer is discarded from the antibody bound beads and 692.8 μl of T4 DNA Polymerase master mix is aliquoted to each of 4 tubes containing beads. 0.2 μl of T4 DNA polymerase (Promega, M4215) is added to the magnetic beads. The DNA polymerase and magnetic beads are mixed and incubated at 37° C. for 40 minutes with rotation on INTELLI-MIXER (program: F8, 30 rpm; U=50, u=60) (http://www.elminorthamerica.com/collections/intelli-mixers/products/elmi-rm-21-intelli-mixers-large-includes-mix-rack). After 40 minutes, tubes are removed from the 37° C. incubator. The T4 DNA polymerase master mix is discarded. The beads are washed with ice-cold wash buffer [PI] three times, and TE once.

### Example 4. dA-Tailing of Chromatin Fragments

The Klenow (3′-5′ exo-) Master Mix contains the following components: Nuclease-free water (616 μl), 10×NEB buffer 2 (70 μl), and 10 mM dATP (7 μl). 7 μl of Klenow Fragment (3′-5′ exo-) and the Klenow Master Mix is added to the beads containing the chromatin fragments. Beads are incubated at 37° C. for 50 minutes. The tubes containing the chromatin fragments are taken out from the 37° C. incubator. The Klenow master mix is decanted from the beads and discarded. The beads are washed with ice-cold wash buffer [PI] for three times, then TE once.

### Example 5. Ligation of Biotin and Fluorescent Label to Barcoded Adaptor

The ligation buffer is prepared containing 1,110 μl nuclease-free water, 4 μl mixed linker, and 280 μl 5×T4 DNA ligase buffer. The ligase buffer is added to the chromatin-containing beads and mixed by flicking. 6 μl T4 DNA ligase are added to the mixture, mixed by flicking, followed by a short spin and a light swirl. The mixture is incubated overnight at 16° C.

### Example 6. Release of Chromatin from Protein G Magnetic Beads

The beads are washed three times with buffer to remove excess linkers. Elution buffer containing % SDS (100 μl 10% SDS+900 μl Buffer TE) is prepared. 200 μl of elution buffer is added to the protein G beads. The tube is placed on the INTELLI-MIXER with rotation (F8, 30 rpm, U=50, u=60) at room temperature for 30 minutes. 200 μl elution buffer-containing chromatin DNA complex from Protein G beads is transferred to a fresh tube. The release reaction is quenched by adding 1.6% of triton X-100 buffer and incubating at 37° C. for 1 hour.

### Example 7. Sample Analysis on the SEQ LL Platform

Prior to addition of chromatin complex, the SEQ LL flow cell surface is blocked with spermine tetrahydrochloride for 1 hour, washed with imaging buffer, and then coated with streptavidin (0.2 mg/ml) for 10 min. The flow cell surface is washed with imaging buffer. The imaging buffer contains 10 mM MES at pH 6.5, 60 mM KCl, 0.32 mM EDTA, 3 mM MgCl₂, 10% glycerol, 0.1 mg/ml actetylated BSA, and 0.02% Igepal. The chromatin complexes are hybridized onto the SEQ LL flow cell surface.

### Example 8. Single Molecule Imaging (SEQ LL)

TIRF microscope with two lasers, 532 nm/75 mW and 640 nm/40 mW, for fluorescence excitation (Compass 215M Cube-40C, Coherent) is used for single molecule sequencing. Both laser beams are filtered through band pass filters (Chroma) and spectrally separated by a dichroic mirror (T: 640 nm, R: 532 nm). The laser beams then pass through the TIRF lens and total internal reflection is achieved through a 60×TIRF oil objective with index of refraction 1.49 (Nikon), and imaged onto a CCD camera. After imaging the chromatin complex, the fluorophore labeled at the linkers is cleaved via addition of TCEP diluted 1:10 in imaging buffer. After incubation with TCEP for 10 min, the flowcell is washed with imaging buffer. All positions are imaged again and residual spots excluded from further analysis (less than 2% of spots remain).

### Example 9. Immunostaining

The antibody specificity dot-blot assay (RNAPII, ChIP grade) is performed by blocking the chromatin bound to the flow cell array with 4 ml of blocking buffer (TBST containing 5% non-fat dried milk) for 4 hours. Next, the flow cell array is washed 3 times with TBST, and primary antibody is added. Antibody is incubated overnight for 4° C. on a rotor. Then the array is washed 3 times in TBST. The second antibody is added for 1 hour at room temperature. The array is washed again 3 times in TBST, and signal is detected by FluorChem Q. (Donkey Anti-Mouse IgG H&L (ALEXA FLUOR 647) pre-adsorbed (ab150111).

Antibodies are diluted in imaging buffer to a final concentration of 50-100 ng/ml, and images are taken every 15 min for total incubation time of 3 hours. (For experiments requiring imaging of more than two marks, the flow cell is washed extensively with imaging buffer (10 washes×5 min incubation for each wash). All positions are imaged again and residual spots excluded from further analysis. Next, the second round of antibodies is applied and imaged as in the first round.

### Example 10. Single Molecule Sequencing

Single molecule scripts are adapted to disable fluidics while imaging flow cell for binding and dissociated events over time. The flow cell is washed with 2M NaCl for 10 minutes, and the temperature increased to 37° C. De-crosslinking for smChIA to release protein from the chromatin complex. The de-crosslinking process may be performed with the following two methods.

Method 1. The flow cells are incubated with proteinase K in TE buffer containing 0.5% SDS buffer at 50° C. overnight or at least 4 hours.

Method 2. The flow cells are incubated with TE buffer containing 0.5% SDS buffer for 65° C. for 2 hours, then incubate with proteinase K in TE buffer containing 0.5% SDS buffer for 4 hours or overnight.

After de-crosslinking, USER restriction enzyme is applied, and incubated for 1 hour at 37° C. The flow cell is washed with several times with H₂O pre-heated to 55° C. Primer is added at a final concentration of 10 nM, the primer is allowed to hybridize for 20 min, following by washing. Single molecule sequencing is then carried out.

### Example 11. Single Molecule ChIP Sequencing (smChIP) Using SEQ LL Platform

In this study, RNAPII ChIP-enriched chromatin materials was prepared from Drosophila S2 cells. Single molecule DNA sequencing from immobilized chromatin was performed on the SEQ LL platform and the obtained smChIP data was compared with previously generated RNAPII ChIA-PET data.

For the smChIA experiments GM12878 or Drosophila S2 cells were single or dual crosslinked with 1% formaldehyde- and 1.5 mM EGS. Crosslinked cells are optionally store at

−80° C. before proceeding to the following steps. Crosslinked S2 cells were subjected to cellular and nuclear lysis. Chromatin fibers were sheared into chromatin complexes by sonication into approximately 3 kb fragments. To the sonicated chromatin complex materials, mixed barcoded linkers were ligated to the DNA in the sheared chromatin complexes (each with distinctive barcodes and biotin), the chromatin sample was then loaded onto a streptavidin-coated flow cell surface, allowing complexes to hybridize and be imaged to determine the density of chromatin on the flow cell. Further, the proteins were removed by de-crosslinking, leaving the immobilized genomic DNA fragments on the surface for sequencing using one primer (P1). In one test run, 16,579 quality reads were generated.

We further tested whether the DNA could be directly sequenced using the SEQ LL platform from the chromatin complexes without the removal of the protein components. A smChIA produced 48,777 reads, demonstrated that high-quality smChIP reads were indeed robustly generated from chromatin complexes directly without first de-crosslinking proteins. The majority of the smChIP reads were ˜30 bp (FIG. 4A).

The mapping of these smChIP reads to the Drosophila reference genome (dm3) showed significant enrichment at the RNAPII binding peaks previously known to be involved in chromatin interactions as identified by RNAPII ChIA-PET (FIG. 4B).

The capability for direct smChIP sequencing from chromatin complexes significantly simplifies the overall smChIA procedure and avoids technical artifacts that may be introduced by protein removal.

### Example 12. Single Molecule Chromatin Interaction Analysis (smChIA)

Following the successful smChIP procedure, sequential sequencing of chromatin complexes using the SEQ LL platform was tested, the core. Chromatin samples were prepared as described above. Rather than using one primer for sequencing, we used two primers (P1 and P2) in series. These primers correspond to two of the linkers ligated to the chromatin DNA fragments immobilized on the flow cell surface. Because each DNA cluster corresponding to a chromatin complex is ˜400 nm in diameter, near the optical resolution of TIRF microscopy, the expected single molecule sequencing reads from the same optical spots have a nucleotide composition consistent with multiple sequencing primers. FIG. 3 shows four DNA templates that were individually sequenced with sequential sequencing using the first primer (P1) followed by the second primer (P2) in series, the P1 sequencing phase generated 16 nt reads, 6 nt from the first primer, P1, and 10 nt from the chromatin DNA, and the P2 sequencing also generated 16 nt reads, 6 nt from the second primer, P2, and 10 nt from a second chromatin DNA fragment. Benefiting from the small Drosophila genome size, most 10 nt chromatin sequences can be uniquely mapped to the Drosophila reference genome, allowing verification that the sequence reads are in fact genomic and elucidating the genomic location of paired interacting loci.

Using traditional ChIA-PET data as a reference, we found many of the smChIA reads fall within pre-defined interaction loci, as RNAPII ChIA-PET previously identified (FIG. 5C.

### Example 13. Development of a Prototype Instrument for smChIA

Based on the smChIA described above, we modified the current SEQ LL platform for smChIA specific requirements. The current SEQ LL platform was designed for whole-genome single molecule DNA and RNA sequencing across 50 channels of flow cells and uses a single laser for fluorescence excitation. This system has been modified for simultaneous detection of histone markers and genomic positions of individual nucleosomes (Shema, et al., (2016) PMID:27151869).

In the smChIA-specific prototype the flow cell footprint size has been reduced to enable rapid experimentation and reduce experimental costs. For the fluidics module, dead volume and footprint size have been reduced. Multicolor imaging using ˜250 mW power lasers has been incorporate 1d into the optics. These modifications significantly reduce cost and allow decreased sequencing chemistry time through use of pairwise-labeled C/T, A/G reversible terminators currently manufactured at SEQ LL. The design is flexible, allowing future incorporation of additional colored lasers. Finally, control software was deployed for imaging and data analysis. The software allows for a range of fields of view to be imaged/sequenced and data to be produced in industry-standard FASTQ format.

### Example 14. smChIA Applied To Single Nuclei (SN)/Single Cell (SC)

The smChIA method does not determine whether multiple distinct chromatin complexes co-exist in an individual single nuclei or different nuclei at the same time. Therefore we extendeded the smChIA technology to the single nucleus level (i.e., single nucleus smChIA). Because a single cell contains a single nucleus, the single nucleus smChIA technology is also coined as single cell smChIA technology.

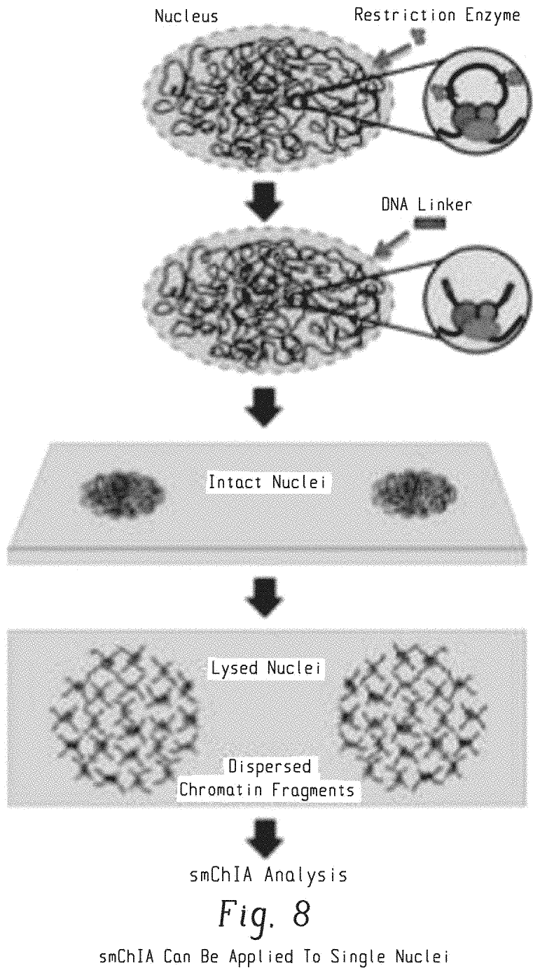

The single nucleus application of the smChIA method takes advantage of existing techniques used to perform in situ permeabilization and restriction enzyme digestion followed by linker ligation (FIG. 8). This allows for the generation of nuclei tagged by linkers genome wide to be directly hybridized to a streptavidin-coated flow cell surface. Importantly, a specific dilution of nuclei must be experimentally determined to ensure single cells are separated from each other such that they may be distinguished as distinct individual nuclei (FIG. 8).

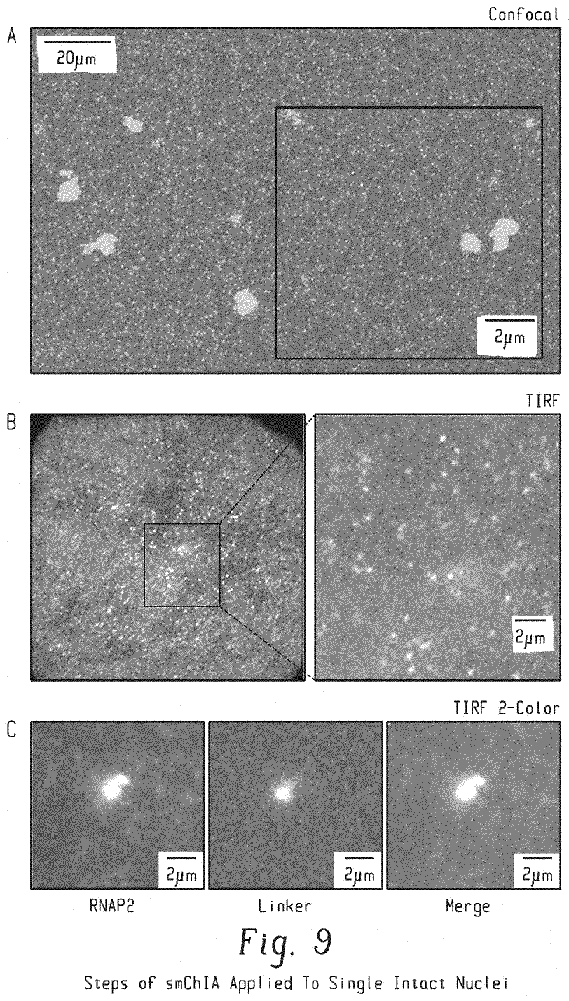

Once single nuclei are hybridized to the flow cell, nuclei can be lysed and the contents dispersed such that single chromatin complexes can be resolved. This method allows for appropriate separation of the individual chromatin complexes without destroying spatial distinction of individual nuclei, such that analysis provides the sequence and protein composition of many single chromatin complexes contained within their respective single nuclei (FIG. 9).

An optimized in situ chromatin digestion procedure was used for the single nucleus smChIA. The cells were first cross-linked by formaldehyde (1%) treatment. The cells were then incubated with a low-salt buffer for cell lysis. Isolated nuclei were solubilized by incubation in SDS (0.5%). To prevent SDS from affecting restriction enzyme efficiency, nuclei were washed with DPBS/0.1% triton X-100. Nuclei were then incubated with MboI (4 bp cutter) at 37° C. overnight.

After washing, the digested chromatin fragments in nuclei were subjected for end-repair and A-Tailing, and then ligated to DNA barcoded linker 1 (P1) with additional fluorescent labeling, such as labeling with ALEXA-647. Confocal microscopic examination showed intact nuclei with strong ALEXA 647 fluorescent signals, indicating that the in situ manipulation of chromatin digestion and linker ligations were successful (FIG. 9A).

Because the nuclei were already permeabilized for in situ digestion and linker ligation, some of the biotin groups ligated to chromatin fragments were exposed on the nuclear surface, thus allowing individual nuclei to be semi-immobilized on the streptavidin-coated slides.

The resulting chromatin preparations of single nuclei were washed with DPBS/0.1% triton X-100 solution, and were examined by TIRF fluorescent microscopy (FIG. 4C).

All publications and patents cited in this specification are herein incorporated by reference in their entirety. Various modifications and variations of the described composition, method, and systems of the invention will be apparent to those skilled in the art without departing from the scope and spirit of the invention. Although the invention has been described in connection with specific preferred embodiments and certain working examples, it should be understood that the invention as claimed should not be unduly limited to such specific embodiments.

1. A method of determining a chromatin interaction at a single molecule level, said method comprising the steps of:
a) crosslinking genomic DNA and proteins in a cell;
b) fragmenting the crosslinked genomic DNA to provide a chromatin complex, the chromatin complex containing DNA and one or more specific proteins;
c) ligating two or more different barcoded linkers to the DNA in the chromatin complex to form a barcoded chromatin complex;
d) immobilizing the barcoded chromatin complex onto a surface;
e) single molecule imaging the barcoded chromatin complex at a single molecule level;
f) sequentially sequencing the DNA in the barcoded chromatin complex at a single molecule level to generate a plurality of sequence reads; and
g) mapping said plurality of sequence reads to a referenced genome to produce a genomic location of said sequenced reads to generate a 3D genomic connectivity map,
wherein said connectivity map is indicative of a physical interaction between the genomic DNA and proteins present in the chromatin complex at a single molecule level.

2. The method of claim 1, wherein prior to immobilizing the barcoded chromatin complex onto the surface, the barcoded chromatin complex is immunoprecipitated by a first antibody capable of binding to the specific protein in the barcoded chromatin complex.

3. The method of claim 1, wherein imaging the barcoded chromatin complex includes immunostaining the barcoded chromatin complex with a second antibody capable of binding to the specific protein in the barcoded chromatin complex.

4. The method of claim 1, wherein prior to immobilizing the barcoded chromatin complex onto the surface, the barcoded chromatin complex is immunoprecipitated by a first antibody capable of binding to the specific protein in the barcoded chromatin complex, and wherein imaging the barcoded chromatin complex includes immunostaining the barcoded chromatin complex with a second antibody capable of binding to the specific protein in the barcoded chromatin complex, and wherein the first antibody and the second antibody are the same.

5. The method of claim 2, wherein fragmenting the crosslinked genomic DNA provides a plurality of chromatin complexes, and the immunoprecipitation enriches the plurality of chromatin complexes for chromatin complexes containing the protein to which the first antibody binds.

6. The method of claim 5, wherein the immunoprecipitation enriches the plurality of chromatin complexes for the chromatin complexes containing the protein to which the first antibody binds by at least a factor of 2 as compared to the plurality of chromatin complexes prior to the immunoprecipitation.

7. The method of claim 1, wherein the barcoded linker contains a fluorescent label, and optionally the barcoded linker additionally contains a biotin molecule and the surface is a streptavidin-coated surface.

8. (canceled)

9. The method of claim 1, wherein the crosslinking is performed in a cell so as to allow the chromatin complex to remain intact in the cell and is followed by permeabilizing the cell.

10. The method of claim 1, wherein the crosslinking step is performed using formaldehyde, EGS, or both.

11-12. (canceled)

13. The method of claim 9, wherein the permeabilizing step is performed using a detergent.

14-15. (canceled)

16. The method of claim 1, wherein the fragmenting step is performing by sonication.

17. The method of claim 1, wherein the fragmenting step is performed by restriction enzyme digestion.

18. The method of claim 2, wherein the barcoded chromatin complex is immunoprecipitated by an antibody capable of binding to a transcription factor or a chromatin architecture factor.

19-20. (canceled)

21. The method of claim 3, wherein the barcoded chromatin complex is immunostained with an antibody capable of binding a transcription factor or a chromatin architecture factor.

22-23. (canceled)

24. The method of claim 1, wherein the chromatin DNA is subjected to end repair and A-Tailing prior to ligating the barcoded linker.

25. The method of claim 1, wherein 2 to 8 different barcoded linkers are ligated to the genomic DNA in the chromatin complex.

26. The method of claim 1, wherein the barcoded linker comprises an oligonucleotide selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

27. The method of claim 1, wherein the barcoded linker comprises a template strand comprising 10-50 nucleotides covalently bound at the 3′-end to a biotin molecule and a non-template strand comprising uracil at multiple loci and fluorescent labeled at the 5′-end.

28. The method of claim 1, further comprising the step of de-crosslinking the barcoded chromatin complex after the barcoded chromatin complex is immobilized on the surface to release the proteins in the chromatin complex.

29. A method of determining a chromatin interaction in a single nucleus, said method comprising the steps of:
a) providing a single nucleus, said nucleus comprising genomic DNA and proteins;
b) crosslinking genomic DNA and proteins in the nucleus;
c) fragmenting the crosslinked genomic DNA in situ to provide a plurality of chromatin complexes, each chromatin complex containing genomic DNA and one or more specific proteins;
d) ligating two or more different barcoded linkers to the DNA in the chromatin complexes, to form barcoded chromatin complexes;
e) immobilizing said single nucleus onto a surface;
f) lysing said single nucleus such that the barcoded chromatin complexes contained in the nucleus are dispersed on the surface;
g) immunostaining the barcoded chromatin complex with an antibody capable of binding to the specific protein present in the barcoded chromatin complex;
h) single molecule imaging the immunostained barcoded chromatin complex;
i) sequential sequencing of the barcoded chromatin complex at a single molecule level to generate a plurality of sequence reads; and
j) mapping the plurality of sequence reads to a referenced genome to produce a genomic location of the sequenced reads to generate a 3D genomic connectivity map;
wherein said connectivity map is indicative a physical interaction between genomic DNA and proteins in the chromatin complex at a single molecule level.

30-57. (canceled)


SEQUENCE LISTING (24 sequences)

SEQ ID NO: 1 - DNA, 33 bp, Artificial Sequence
  Synthetic Oligonucleotide
ttaaccgatcacttctacgataccaaagtatct

SEQ ID NO: 2 - DNA, 32 bp, Artificial Sequence
  Synthetic Oligonucleotide
atcacgactcatggtataacacacctatatct

SEQ ID NO: 3 - DNA, 32 bp, Artificial Sequence
  Synthetic Linker
cgatgtagattcatcgtaccatactactatct

SEQ ID NO: 4 - DNA, 32 bp, Artificial Sequence
  Synthetic Linker
ttaggcagtatacacagtactactcagcatct

SEQ ID NO: 5 - DNA, 33 bp, Artificial Sequence
  Synthetic Linker
tgaccaatcacaactataactgatgacacatct

SEQ ID NO: 6 - DNA, 32 bp, Artificial Sequence
  Synthetic Linker
acagtgatgatacgacgagtactgagatatct

SEQ ID NO: 7 - DNA, 32 bp, Artificial Sequence
  Synthetic Linker
gccaatatagagcatactacagaagacgatct

SEQ ID NO: 8 - DNA, 32 bp, Artificial Sequence
  Synthetic Linker
cagatcagtcagtgatatcacgactgacatct

SEQ ID NO: 9 - DNA, 34 bp, Artificial Sequence
  Synthetic Linker
agauacutugguatcguagaaguguacggttaat

SEQ ID NO: 10 - DNA, 33 bp, Artificial Sequence
  Synthetic Linker
agauataggugtgutauaccaugagucgtgatt

SEQ ID NO: 11 - DNA, 33 bp, Artificial Sequence
  Synthetic Oligonucleotide
agauaguagtaugguacgaugaatcuacatcgt

SEQ ID NO: 12 - DNA, 33 bp, Artificial Sequence
  Synthetic Oligonucleotide
agaugcugaguagtacugtguatacugcctaat

SEQ ID NO: 13 - DNA, 33 bp, Artificial Sequence
  Synthetic Oligonucleotide
agaugtgucaucagutauagtgugautggtcat

SEQ ID NO: 14 - DNA, 33 bp, Artificial Sequence
  Synthetic Oligonucleotide
agauatcucaguacucgucguatcaucactgtt

SEQ ID NO: 15 - DNA, 33 bp, Artificial Sequence
  Synthetic Oligonucleotide
agaucgtcutcugtaguatgcuctauattggct

SEQ ID NO: 16 - DNA, 33 bp, Artificial Sequence
  Synthetic Oligonucleotide
agaugucagucgugataucacugacugatctgt

SEQ ID NO: 17 - DNA, 26 bp, Artificial Sequence
  Synthetic Oligonucleotide
agatactttggtatcgtagaagtgat

SEQ ID NO: 18 - DNA, 26 bp, Artificial Sequence
  Synthetic Oligonucleotide
agatataggtgtgttataccatgagt

SEQ ID NO: 19 - DNA, 26 bp, Artificial Sequence
  Synthetic Oligonucleotide
agatagtagtatggtacgatgaatct

SEQ ID NO: 20 - DNA, 26 bp, Artificial Sequence
  Synthetic Oligonucleotide
agatgctgagtagtactgtgtatact

SEQ ID NO: 21 - DNA, 26 bp, Artificial Sequence
  Synthetic Oligonucleotide
agatgtgtcatcagttatagtgtgat

SEQ ID NO: 22 - DNA, 26 bp, Artificial Sequence
  Synthetic Oligonucleotide
agatatctcagtactcgtcgtatcat

SEQ ID NO: 23 - DNA, 26 bp, Artificial Sequence
  Synthetic Oligonucleotide
agatcgtcttctgtagtatgctctat

SEQ ID NO: 24 - DNA, 26 bp, Artificial Sequence
  Synthetic Oligonucleotide
agatgtcagtcgtgatatcactgact